(19) United States
(12) Patent Application Publication
(10) Pub. No.: US 2004/0068050 A1
(43) Pub. Date: Apr. 8, 2004

(54) Homogeneous microemulsion comprising polyethylene glycol

(75) Inventors: Dennis Miller, Kelkheim (DE); Torsten Henning, Bad Soden (DE)

Correspondence Address: Clariant Corporation, Industrial, Property Department,, 4000 Moroe Road, Charlotte, NC, 28205, US

(73) Assignee: Clariant GmbH

(21) Appl. No.: 10/680,852

(22) Filed: Oct. 7, 2003

(30) Foreign Application Priority Data
Oct. 7, 2002 (DE) 10246706.4

(51) Int. Cl. A61K 31/74; C08K 3/00

(52) U.S. Cl. 524/612; 424/78.02

(57) ABSTRACT

A homogeneous, transparent microemulsion comprises

a) 1 to 70% by weight of a water-insoluble liquid;

b) 1 to 98% by weight of a polyethylene glycol;

c) 0 to 97% by weight of water; and

d) 1 to 20% by weight of a surfactant

The microemulsion is thermodynamically stable and is preferably suitable for the preparation of cosmetic compositions, preferably of bath oil, or of cleaning compositions.

The present invention relates to a homogeneous, transparent microemulsion, which comprises polyethylene glycol.

The phase behavior of hydrocarbon/surfactantlwater systems is the subject of numerous investigations. The appropriate choice of individual components results in microemulsions being obtained in which relatively large amounts of hydrocarbon are solubilized in the aqueous medium. Such microemulsions are transparent, where the terms transparent and translucent are used synonymously, and are described, for example, in

H. G. Hauthal and K. Quitsch “Neues uber Mikcroemulsionen” [New aspects of microemulsions], Z. Chem., 30, 274-281 (1990).

Similar phenomena as with the microemulsions are also observed with some other nonpolar liquids. In the literature, the nonpolar liquid is generally described using the term “oil” or “oil phase”.

In practice, the formulation of a microemulsion is often difficult. To form the microemulsion, the surfactant must be optimized for the system in question. This is done, for example, by changing the length of the polyethylene oxide chain of an oxethylate. In addition, an adequate amount of surfactant must be present in order to solubilize the oil. The larger the oil molecules, the more difficult it is to formulate a microemulsion. Depending on the composition, the addition of a shortchain amphiphile (cosurfactant), such as, for example, butanol, may be necessary. Finally, some formulations are only stable over a limited temperature range.

Surprisingly, it has been found that by completely or partially replacing the water with polyethylene glycol (PEG), the formation of a microemulsion can be enabled or at least eased.

The invention therefore provides a microemulsion of the type mentioned at the beginning whose characteristic feature is to be regarded as being the fact that it comprises the following constituents:

a) 1 to 70% by weight, preferably 10 to 50% by weight, of a water-insoluble liquid;

b) 1 to 98% by weight, preferably 20 to 80% by weight, of a polyethylene glycol;

c) 0 to 97% by weight, preferably 0 to 60% by weight, of water; and

d) 1 to 20% by weight, preferably 2 to 15% by weight, of a surfactant with the chemical formula:

R—(OCHR′CH)ₙOH,

in which R is an alkyl or alkenyl radical having 8 to 22 carbon atoms, preferably having 12 to 18 carbon atoms, or is an alkylphenol or polyalkylphenol radical having 4 to 16 alkyl carbon atoms,

in which R′ is H or CH₃ or a mixture thereof, preferably H,

and in which n is an integer in the range from 2 to 20,

where all of the data in % by weight are based on the total weight of the microemulsion.

In the microemulsion according to the invention, if the minimum amount of surfactant, component d), is reduced as much as possible, the addition of a cosurfactant, a short-chain alcohol, is additionally not necessary. The advantage of the use of PEG may also be that the microemulsion is formed over a relatively broad HLB range (=hydrophilic/lipophilic balance value) of the surfactant, resulting in a significantly greater freedom when working out the formulation.

Polyethylene glycols which are suitable as component b) for the microemulsion according to the invention preferably have an average molar mass in the range from 150 to 35 000 g/mol, preferably from 200 to 800 g/mol, measured by determining OH numbers from 600 to 1 mg of KOH/g, preferred range: 591 to 134 mg of KOH/g.

The surfactants which are suitable as component d) for the microemulsion according to the invention may be nonionic, cationic, anionic and/or amphoteric surfactants.

The nonionic surfactants are preferably fatty alcohol ethoxylates, dimethylamine oxides, ethoxylated castor oils, alkyl polyglucosides, fatty acid sorbitol esters, fatty acid polyglycerol esters, ethoxylated fatty acid polyglycerol esters, fatty acid monoethanolamide ethoxylates, glycerol monoesters and diesters of fatty acids and/or phosphoric triesters.

Likewise preferred nonionic surfactants are (C₈-C₂₂)-alkyl or alkenyl ethoxylates with 2 to 20 ethylene oxide groups.

The anionic surfactants are preferably phosphoric monoesters, phosphoric diesters, alkyl sulfates, alkyl ether sulfates, preferably sodium laureth sulfate, alkylamidopolyglycol ether sulfates, alkylpolyglycol ether carboxylates, alkylpolyglycol ether sulfosuccinates and/or fatty acid isethionates.

The amphoteric surfactants are preferably acyl glutamates, alkylamidopropylbetaines, preferably cocoamidopropylbetaine, fatty acid methyl taurides, fatty acid sarcosides and/or amphoacetates.

In a particular embodiment, the surfactants are betaines, alkyl ether sulfates or mixtures thereof.

Suitable water-insoluble liquids which are suitable as component a) for the microemulsion according to the invention are preferably mineral oils, polydecenes, triglycerides, e.g. capric/caprylic triglycerides, natural oils, e.g. orange oil, and/or esters, preferably stearates, palmitates and myristates.

The water-insoluble liquid of component a) is preferably solubilized in the microemulsion according to the invention with a degree of solubilization S greater than or equal to 0.8, preferably greater than or equal to 1.5. The degree of solubilization S is the volume ratio of component a) to the surfactant of component d).

In a particular embodiment, the microemulsion can also additionally comprise polar organic compounds, preferably hydroxy compounds and/or polyhydroxy compounds, particularly preferably glycerol, propylene glycol, ethanol, hexylene glycol and/or isopropanol, in the amounts suitable in each case.

In a further particular embodiment, the microemulsion can also comprse water-soluble and/or oil-soluble, preferably differently colored, dyes. This allows advantageous optical effects of an esthetic nature to be achieved.

The microemulsions according to the invention are particularly suitable for cosmetic formulations and household formulations. A particular advantage here is that polyethylene glycol, in contrast to some other cosurfactants, is neither toxic nor flammable.

In the cosmetics sector, the microemulsions have the following applications:

bath oils and shower gels with refatting action;

special skincare compositions;

solubilization of active ingredients, e.g. in sunscreen compositions.

Depending on the intended use, cosmetic formulations can also comprise further constituents, such as, for example, plant extracts, vitamins, antioxidants, hydroxycarboxylic acids, fatty alcohols, allantoin, thickeners or consistency-imparting agents, organic or inorganic UV absorbers, perfume, preservatives, dyes, pearlizing agents, cationic polymers, organosilicones, (e.g. caprylyl trimethicone, phenyltrimethicone) and pH regulators.

The microemulsions according to the invention are also suitable as cleaning compositions, e.g. for particularly stubborn soiling on hard surfaces, or for the pretreatment of stains prior to the washing of items of clothing.

Depending on the intended use, cleaning compositions comprise further constituents, such as, for example, sodium citrate, zeolites, inorganic phosphates, complexing agents, abrasive substances, polyacrylic acids and salts thereof, color protection additives, acids or alkalis, triethanolamine, electrolytes, optical brighteners, thickeners, perfume, dyes, preservatives, solvents, enzymes, disinfectants and antifoams.

Further applications of the microemulsions according to the invention are:

solubilization of active ingredients in pharmaceutical and agrochemical formulations;

special solution media for preparative and analytical chemistry.

In some technical applications, it is desirable to introduce as much oil as possible with as little surfactant as possible into the microemulsion. The maximum volume ratio of oil to surfactant, which is defined as the degree of solubilization S of the oil, can be taken as the measure of the effectiveness of the surfactant. S depends inter alia on the chemical composition of the oil. However, this analysis is only appropriate if the volume of the oil is less than the volume of the polar liquid (here PEG/water mixture).

The microemulsion according to the invention can advantageously be prepared by simply mixing the individual components a), b), optionally c) and d).

The thermodynamic equilibrium of the microemulsion is automatically established following mixing.

In a preferred embodiment, the hydrophilic components (polyethylene glycols, water, water-soluble dyes etc.), including the surfactants, are mixed together. Separately from this, the hydrophobic, water-insoluble components (oils, oil-soluble dyes etc.) are mixed together. The hydrophilic mixture and the hydrophobic mixture are then mixed together.

### WORKING EXAMPLES

Examples 1 and 2 show microemulsions according to the invention.

Oleth-5, Oleth-8 and Oleth-10 are the INCI names (INCI=International Nomenclature of Cosmetic Ingredients) for oleyl alcohol polyglycol ethers with 5 or 8 or 10 mol of ethylene oxide.

PEG-8 is the INCI name for polyethylene glycol with an average molar mass of 400. All of the quantitative data are in % by weight.

TABLE 1

| Example 1 | | Example 2 | |
|---|---|---|---|
| Component | % by wt. | Component | % by wt. |
| Paraffin oil | 38.6 | Decane | 36.9 |
| Oleth-5 | 6.5 | Oleth-8 | 10.1 |
| Oleth-8 | 6.5 | Oleth-10 | 1.4 |
| PEG-8 | 19.4 | PEG-8 | 20.6 |
| Water | 29.0 | Water | 30.9 |
| Appearance | Homogeneous | Appearance | Homogeneous |
| | transparent liquid | | transparent liquid |

The compositions of examples 1 and 2 listed above were converted to proportions by volume so that the effectiveness of the surfactant can be assessed more precisely. The result is given below in table 2.

TABLE 2

| | Example 1 | Example 2 |
|---|---|---|
| Component | % by vol. | % by vol. |
| Oil | 43.7 | 44.7 |
| Polar liquid (PEG/water) | 43.7 | 44.7 |
| Surfactant | 12.6 | 10.6 |

In the case of example 2, the degree of EO (=degree of ethoxylation) was varied in additional experiments by changing the ratio of Oleth-8: Oleth-10 or Oleth-5: Oleth-8. The phase behavior was also investigated at different temperatures. In this connection, it has been found that the phase behavior, which is a sign of the stability of the microemulsion, is largely independent of the temperature.

### Example 3

This example gives in each case comparative measurements with and without PEG. Tests were performed as to whether there is an optimum range of the degree of EO in which a microemulsion is formed. In order to vary the degree of EO, mixtures of surfactants with different degrees of EO were used as appropriate (Oleth-2, Oleth-5, Oleth-8 and Oleth-10).

Microemulsion according to the invention (percentages in % by weight, based on total weight):

| | |
|---|---|
| Paraffin oil | 16.0% |
| Capric/Caprylic triglyceride | 4.0% |
| Oleth-5 | 4.0% |
| Oleth-8 | 4.0% |
| PEG-8 | 28.8% |
| Water | 43.2% |

The mixture formed a homogeneous transparent liquid.

### Comparative Experiments

In a comparative experiment, the PEG-8 was replaced with water. The average degree of EO of the surfactant was varied from 2 to 10. No formation of microemulsions was observed.

1. A homogeneous, transparent microemulsion which comprises polyethylene glycol and:
a) 1 to 70% by weight, preferably 10 to 50% by weight, of a water-insoluble liquid;
b) 1 to 98% by weight, preferably 20 to 80% by weight, of a polyethylene glycol;
c) 0 to 97% by weight, preferably 0 to 60% by weight, of water; and
d) 1 to 20% by weight, preferably 2 to 15% by weight, of a surfactant with the chemical formula:
R—(OCHR′CH)ₙOH,
in which R is an alkyl or alkenyl radical having 8 to 22 carbon atoms, preferably having 12 to 18 carbon atoms, or is an alkylphenol or polyalkylphenol radical having 4 to 16 alkyl carbon atoms, in which R′ is H or CH₃ or a mixture thereof, preferably H, and in which n is an integer in the range from 2 to 20,
where all of the data in % by weight are based on the total weight of the microemulsion.

2. The microemulsion as claimed in claim 1, wherein the weight ratio of polyethylene glycol:water is in the range from 99:1 to 30:70.

3. The microemulsion as claimed in claim 1 or 2, characterized in that the water-insoluble liquid is less than 50% by volume of the microemulsion and the volume ratio of water-insoluble liquid to surfactant is at least 2.0.

4. The microemulsion as claimed in one or more of claims 1 to 3, wherein the polyethylene glycol has a molar mass in the range from 150 to 35 000 g/mol, preferably from 200 to 800 g/mol.

5. The microemulsion as claimed in one or more of claims 1 to 4, which comprises, as component a), the water-insoluble liquid, oils, hydrocarbons, preferably mineral oil or polydecenes, triglycerides, preferably natural oils and/or esters, preferably stearates, palmitates and myristates.

6. The microemulsion as claimed in one or more of claims 1 to 5, which comprises, as component d) a surfactant chosen from nonionic, cationic, anionic and amphoteric surfactants.

7. The microemulsion as claimed in claim 6, which comprises, as nonionic surfactant, dimethylamine oxides, ethoxylated castor oils, poloxamers, alkyl polyglucosides, fatty acid sorbitol esters, fatty acid polyglycerol esters, ethoxylated fatty acid polyglycerol esters, fatty amine ethoxylates, fatty acid monoethanolamide ethoxylates, glycerol mono- and diesters of fatty acids and/or phosphoric triesters.

8. The microemulsion as claimed in claim 6, which comprises, as anionic surfactant, phosphoric monoesters, phosphoric diesters, alkyl sulfates, alkylbenzenesulfonates, alkanesulfonates, alkylpolyglycol ether sulfates, preferably sodium laureth sulfate, alkylamidopolyglycol ether sulfates, alkylpolyglycol ether carboxylates, alkylpolyglycol ether sulfosuccinates and/or fatty acid isethionates.

9. The microemulsion as claimed in claim 6, which comprises, as amphoteric surfactant, acyl glutamate, alkylamidopropylbetaines, preferably cocoamidopropylbetaine, fatty acid methyl taurides, fatty acid sarcosides and/or amphoacetates.

10. The microemulsion as claimed in one or more of claims 1 to 9, wherein component a) is solubilized in the microemulsion with a degree of solubilization S greater than or equal to 0.8, preferably greater than or equal to 1.5.

11. The microemulsion as claimed in one or more of claims 1 to 10, which additionally comprises an electrolyte.

12. The microemulsion as claimed in one or more of claims 1 to 11, which additionally comprises polar organic compounds, preferably hydroxy compounds and/or polyhydroxy compounds, particularly preferably glycerol, propylene glycol, ethanol and/or isopropanol.

13. The use of a microemulsion as claimed in one or more of claims 1 to 12 for cosmetic formulations.

14. The use of a microemulsion as claimed in one or more of claims 1 to 12 for cleaning compositions.
